(19) United States
(12) Patent Application Publication
(10) Pub. No.: US 2008/0003252 A1
(43) Pub. Date: Jan. 3, 2008

(54) Functionalized hydrophilic macromonomers and medical devices incorporating same

(76) Inventors: Yu-Chin Lai, Pittsford, NY (US); Weihong Lang, Penfield, NY (US)

Correspondence Address: Bausch & Lomb Incorporated, One Bausch & Lomb Place, Rochester, NY, 14604-2701, US

(21) Appl. No.: 11/479,227

(22) Filed: Jun. 30, 2006

(51) Int. Cl. A61F 2/02; C08L 53/00

(52) U.S. Cl. 424/423; 525/92 C; 424/428

(57) ABSTRACT

A macromonomer of the present invention comprises at least functional group and a plurality of hydrophilic moieties. Such a functional group can be an ethylenically unsaturated group that comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group. Polymeric materials comprise such a macromonomer are used advantageously to provide a hydrophilic or lubricious (or both) coating on polymeric articles, such as medical devices. Such polymeric materials can comprise units of other hydrophilic monomers.

### BACKGROUND OF THE INVENTION

The present invention relates to functionalized hydrophilic macromonomers. In particular, the present invention relates to such macromonomers comprising a plurality of hydrophilic moieties and medical devices comprising the same.

Advances in the chemistry of materials for medical devices have increased the comfort for their extended use in a body environment. Furthermore, extended use of medical devices, such as ophthalmic lenses, has become increasingly favored due to the availability of soft contact lenses having high oxygen permeability (e.g., exhibiting high Dk values greater than 80) and/or high water content. Such lenses are increasingly made of silicone-containing materials. Although these materials have some desirable properties for ophthalmic applications, they tend to have relatively hydrophobic surfaces that have a high affinity for lipids and proteins. Accumulation of these materials can interfere with the clarity of the lens and the comfort of the wearer. On the other hand, hydrophilic surfaces tend to limit the adsorption onto and absorption into ophthalmic lenses of tear lipids and proteins and allow the lenses to move relatively freely on the eye, thus providing increased comfort to the wearer.

A known method for modifying the surface hydrophilicity of a relatively hydrophobic ophthalmic device, such as a contact lens, is through the use of a plasma treatment. Plasma treatment techniques are disclosed, for example, in PCT Publications WO 96/31792 to Nicolson et al., WO 99/57581 to Chabrececk et al., and WO 94/06485 to Chatelier et al. In the Chabrececk et al. application, photoinitiator molecules are covalently bound to the surface of the article after the article has been subjected to a plasma treatment which provides the surface with functional groups. A layer of polymerizable macromonomer is then coated onto the modified surface and heat or radiation is applied to graft polymerize the macromonomer to form the hydrophilic surface. However, it may be difficult to provide an effective number of photoinitiators on the surface to effect a strong attachment of the resulting polymer.

Other methods of permanently altering the surface properties of polymeric biomaterials, such as contact lenses, have been developed. Some of these techniques include Langmuir-Blodgett deposition, controlled spin casting, chemisorptions, and vapor deposition. Examples of Langmuir-Blodgett layer systems are disclosed in U.S. Pat. Nos. 4,941,997; 4,973,429; and 5,068,318. Like plasma treatments, these techniques are not cost-effective methods that may easily be incorporated into automated production processes for making biomedical devices such as contact lenses.

Another method of producing a hydrophilic surface is discussed in U.S. Pat. No. 6,926,965. The method is carried out in a layer-by-layer (LbL) fashion, which involves consecutively dipping a substrate into oppositely charged polymeric materials until a coating of a desired thickness is formed.

These prior-art methods are tedious. As a result, the manufacturing costs for the finished devices can be high.

Therefore, there is a continued need to provide materials that enable or facilitate the manufacture of medical devices, such as ophthalmic lenses, that have improved hydrophilic surfaces and are compatible with physiological environment, and improved methods for making them.

### SUMMARY OF THE INVENTION

In general, the present invention provides functionalized hydrophilic macromonomers, articles comprising such macromonomers, and methods of making and using the same.

In one aspect, a functionalized macromonomer of the present invention comprises at least an ethylenically unsaturated group and a plurality of hydrophilic moieties.

In another aspect, such an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group.

In still another aspect, such an ethylenically unsaturated group can be further converted to a different functional group that permits the macromonomer to under chemical reactions with other functionalized materials.

In still another aspect, the present invention provides a polymer comprising units of such a macromonomer and units of at least an additional monomer.

In still another aspect, such an additional monomer comprises at least a hydrophilic moiety.

In still another aspect, the present invention provides a polymeric article, a surface of which is modified with a material comprising units of a macromonomer that comprises both at least a terminal α,β-conjugated carboxylic group (or an α,β-conjugated carboxylic group along the chain of the macromonomer) and a plurality of hydrophilic groups.

In still another aspect, the polymeric article is a medical device.

In still another aspect, the medical devices are ophthalmic devices.

In yet another aspect, the medical devices are contact lenses.

In still another aspect, the medical device modified with a macromonomer of the present invention has a lubricious surface and provides enhanced comfort to a user.

In a further aspect, the present invention provides a method for making an ethylenically unsaturated hydrophilic macromonomer. The method comprises reacting an oligomer or polymer having a first functional group with a compound having a second functional group that is capable of reacting with the first functional group, wherein the compound comprises an ethylenically unsaturated group.

In yet another aspect, the present invention provides a method of making a medical device that has a hydrophilic or lubricious (or both) surface. The method comprises: (a) providing the medical device having at least a medical-device surface functional group; (b) providing a material comprising units of a macromonomer having an ethylenically unsaturated group and a plurality of hydrophilic moieties, said ethylenically unsaturated group being capable of interacting with said at least a medical-device surface functional group; and (c) contacting the medical device with the material at a condition sufficient to produce the medical device having an increased surface hydrophilicity or lubricity or both.

Other features and advantages of the present invention will become apparent from the following detailed description and claims.

### DETAILED DESCRIPTION OF THE INVENTION

In general, the present invention provides functionalized hydrophilic macromonomers.

In one aspect, such a macromonomer has an ethylenically unsaturated group.

In another aspect, a macromonomer of the present invention comprises at least an ethylenically unsaturated group and a plurality of hydrophilic moieties. In one embodiment, said at least an ethylenically unsaturated group is a terminal group. In another embodiment, the macromonomer has both terminal ethylenically unsaturated groups. In still another embodiment, said at least an ethylenically unsaturated group is or comprises a part of a pendant group attached to a portion of the macromonomer.

In another aspect, such an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group. In some embodiments, such an ethylenically unsaturated group comprises acrylate, methacrylate, maleate, fumarate, or itaconate group.

In still another aspect, the macromonomer comprises units of a hydrophilic monomer that include such hydrophilic groups.

In still another aspect, such a macromonomer has a formula of

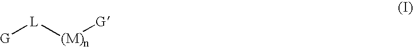

wherein G and G′ are groups that comprise at least an ethylenically unsaturated group; L is a direct bond or a divalent linkage group that comprises a hydrocarbon group or a heterohydrocarbon group; M represents a hydrophilic monomeric unit; and n is a positive integer in the range from about 2 to about 1000. In some embodiments, n is in the range from about 2 to about 800, or from about 2 to about 600, or from about 10 to about 600, or from about 20 to about 600, or from about 20 to 500. In one embodiment, L is a direct bond. In another embodiment, L comprises a C₁₋₁₀ linear saturated or unsaturated hydrocarbon group, a C₃₋₁₀ branched saturated or unsaturated hydrocarbon group, or a C₃₋₁₀ cyclic saturated or unsaturated hydrocarbon group. In still another embodiment, L also can include one or more atoms selected from the group consisting of O, N, S, and combinations thereof.

In some embodiments, G′ can be absent, and a macromonomer of the present invention has a formula of

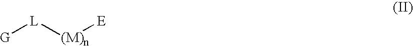

wherein G, L, M, and n have the meanings as disclosed above, and E comprises a C₁₋₁₀ linear or branched hydrocarbon group, or a C₃₋₁₀ cyclic saturated, or unsaturated hydrocarbon group, or a heteroatom.

In still another aspect, G and G′ are independently selected from the group consisting of acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, and styryl groups.

In still another aspect, (M)ₙ represents oligomeric or polymeric chain comprising units of N-vinylpyrrolidone. In alternative embodiments, (M)ₙ represents oligomeric or polymeric chain comprising units of polyhydric alcohols (such as glyceryl methacrylate or glyceryl acrylate), dimethyl methacrylamide, dimethyl acrylamide (“DMA”), 2-hydroxyethyl methacrylate (“HEMA”), 2-hydroxyethyl acrylate, erythritol (meth)acrylate, xylitol (meth)acrylate, sorbitol (meth)acrylate, or derivatives thereof. The term “(meth)acrylate” means methacrylate or acrylate. In some other embodiments, (M)ₙ represents oligomeric or polymeric chain comprising units of one of the foregoing monomers and units of an alkylene oxide (such as ethylene oxide or propylene oxide). Still other non-limiting examples of M include acrylic acid, methacrylic acid, and Zwitterionic monomer such as {3-(methacryloylamino)propyl}dimethyl(3-sulfopropyl)ammonium hydroxide inner salt.

In one embodiment, a macromonomer of the present invention is a methacrylate- or acrylate-terminated poly(N-vinylpyrrolidone). Poly(N-vinylpyrrolidone) is sometimes herein abbreviated as “PVP”. Such a macromonomer is represented by the following formula.

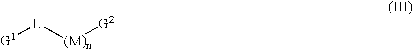

wherein G¹ and G² are independently selected from the group consisting of acrylate and methacrylate; G² can be absent in some embodiments; and L, M, and have the meanings disclosed above.

In a further aspect, the present invention provides a method for making an ethylenically unsaturated hydrophilic macromonomer. The method comprises reacting an oligomer or polymer having a first functional group with a compound having a second functional group that is capable of reacting with the first functional group, wherein the compound comprises an ethylenically unsaturated group.

An acrylate- or methacrylate-terminated PVP can be prepared by reacting acryloyl chloride or methacryloyl chloride with monohydroxy- or dihydroxy-terminated PVP in the presence of a weak base, such as a tertiary amine or an alkali carbonate. For example, an acrylate-terminated PVP is prepared according to the following reaction.

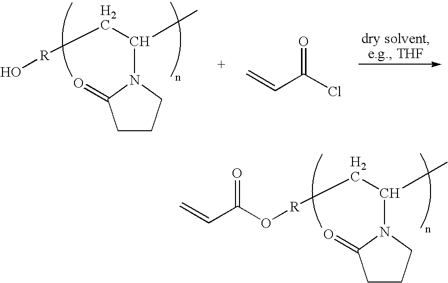

wherein R is a direct bond or a divalent group selected from the group consisting of C₁₋₁₀ saturated and unsaturated hydrocarbon groups, C₁₋₁₀ saturated and unsaturated hydrocarbon groups having one or more heteroatoms therein, C₃₋₁₀ cyclic hydrocarbon groups, C₃₋₁₀ heterocyclic groups, C₆₋₃₆ aryl groups, and C₆₋₃₆ heteroaryl groups. In some embodiments, R is a C₁₋₁₀ alkyl group. In some other embodiment, R is methylene, dimethylene, trimethylene, or tetra methylene. Although acryloyl and methacryloyl chloride are disclosed above, other acryloyl and methacryloyl halides are also useable.

Alternatively, hydroxy-terminated PVP can be reacted with glycidyl acrylate or glycidyl methacylate to yield acrylate- or methacrylate-terminated PVP. For example, methacrylate-terminated PVP can be prepared according to the following reaction, wherein R has the meaning disclosed above.

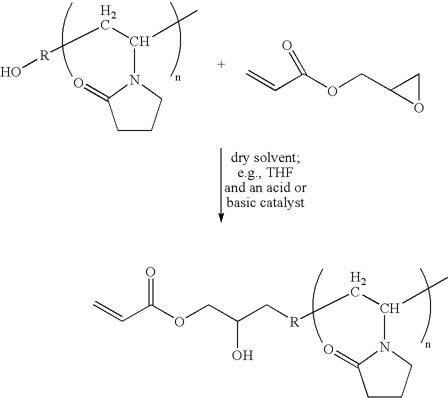

In another embodiment, hydroxyl-terminated PVP can be reacted with 2-isocyantoethyl acrylate or 2-isocyanato methacylate to yield acrylate- or methacrylate-terminated PVP. For example, methacrylate-terminated PVP is prepared according to the following reaction, wherein R has the meaning disclosed above.

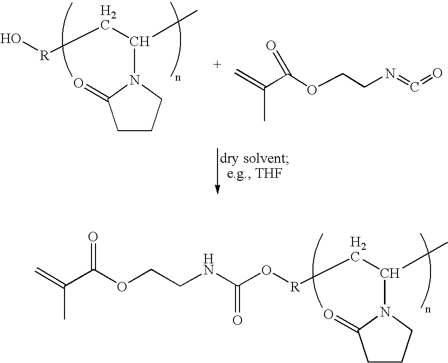

In still another embodiment, such a methacrylate- or acrylate-terminated PVP can be further modified to produce amino-terminated PVP, for example, according to the following scheme.

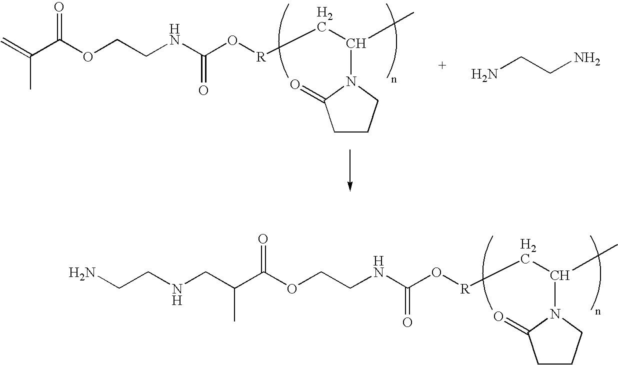

In another embodiment, vinyl-, allyl-, or styryl-terminated PVP can be prepared by reacting hydroxy-terminated PVP with vinyl, allyl, or styryl isocyanate, respectively. For example, vinyl-terminated can be prepared from hydroxyl-terminated PVP and vinyl isocyanate according to the following reaction, wherein R has the meaning disclosed above.

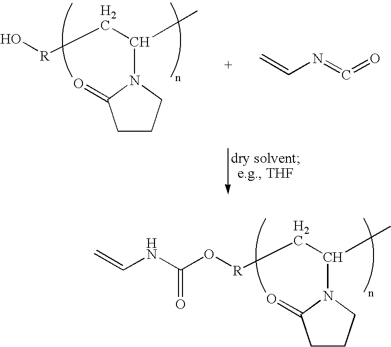

In still another embodiment, a maleate- or itaconate-terminated PVP can be prepared by reacting maleic anhydride or itaconic anhydride with hydroxyl-terminated PVP. In still another embodiment, fumarate-terminated PVP can be prepared by reacting fumaric acid with hydroxyl-terminated PVP. For example, maleate-terminated PVP can be prepared according to the following reaction, wherein R has the meaning disclosed above.

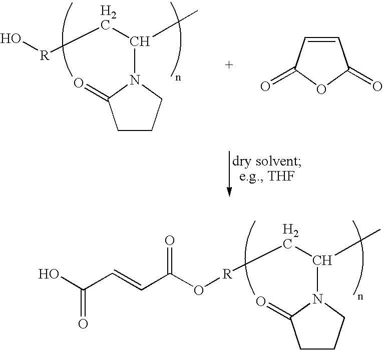

It should be noted that a macromonomer comprising hydrophilic moieties other than, or in addition to N-vinylpyrrolidone, can be prepared in a method similar to one of those disclosed above. Non-limiting examples of other hydrophilic monomers comprising such other hydrophilic moieties include glyceryl (meth)acrylate, dimethyl (meth)acrylamide, 2-hydroxyethyl (meth)acrylate, erythritol (meth)acrylate, xylitol (meth)acrylate, sorbitol (meth)acrylate, derivatives thereof, combinations thereof, and mixtures thereof. The term “(meth)acrylate” means methacrylate or acrylate and “(meth)acrylamide” means methacrylamide or acrylamide. It should be noted that a macromonomer of the present invention can comprise units of one or more hydrophilic monomers disclosed above.

In another aspect, a macromonomer comprising units of the present invention can be used to provide a coating on a polymeric article, which coating renders the polymeric article more hydrophilic and/or lubricious. An ethylenically unsaturated group of a macromonomer of the present invention can react with a complementary group on a surface of the polymeric article to yield a coating comprising units of the macromonomer.

In one aspect, the polymeric article is a medical device. In one embodiment, the medical device is an ophthalmic device. In another embodiment, the ophthalmic device is a contact lens.

In a further aspect, medical articles that are in contact with body fluid, such as a wound dressing, catheters, implants (e.g., artificial hearts or other artificial organs), can be provided with a hydrophilic coating comprising a macromonomer of the present invention to inhibit bacterial attachment and growth or to reduce a deposit of lipids or proteins thereon.

For example, in one embodiment of the present invention, wherein a contact lens comprises surface amino group, and the coating macromonomer comprises acrylate-terminated poly(N-vinylpyrrolidone), the surface-modified contact lens is produced according to Scheme 1, wherein n is a positive integer and R has the meaning disclosed above. In one embodiment, n can range from about 5 to about 1000, or from about 10 to 800, or from about 10 to about 600, or from about 20 to about 500.

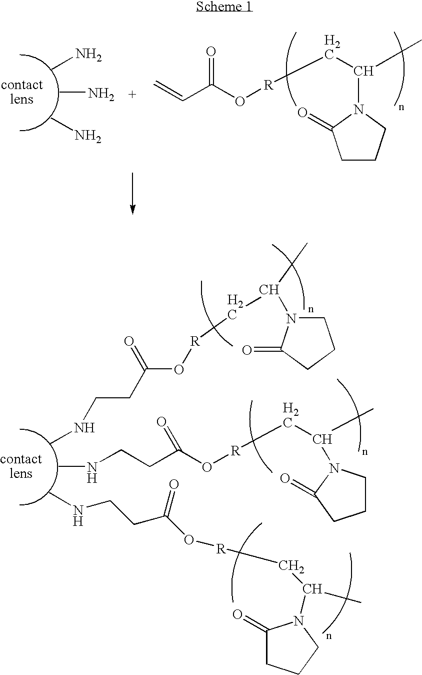

In another embodiment of the present invention, wherein a contact lens comprises surface hydride group (e.g., Si—H group), and the coating macromonomer comprises vinyl-terminated poly(N-vinylpyrrolidone), the surface-modified contact lens is produced according to Scheme 2, wherein n is a positive integer and R has the meaning disclosed above. In one embodiment, n can range from about 5 to about 1000, or from about 10 to 800, or from about 10 to about 600, or from about 20 to about 500.

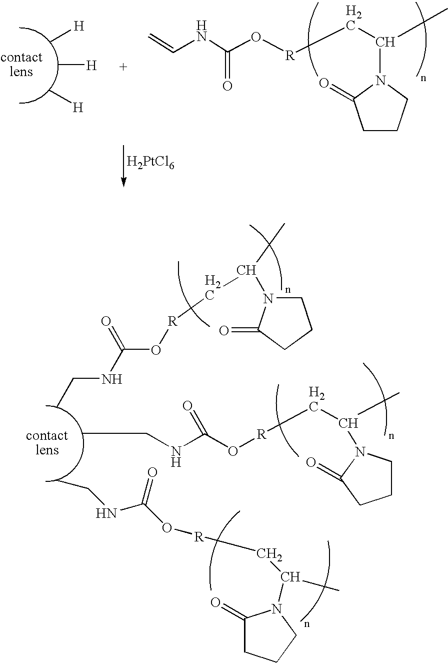

In another embodiment of the present invention, wherein a contact lens comprises surface amino group, and the coating macromonomer comprises acrylate-terminated poly(glyceryl methacrylate), the surface-modified contact lens is produced according to Scheme 3, wherein n is a positive integer and R has the meaning disclosed above. In one embodiment, n can range from about 5 to about 1000, or from about 10 to 800, or from about 10 to about 600, or from about 20 to about 500.

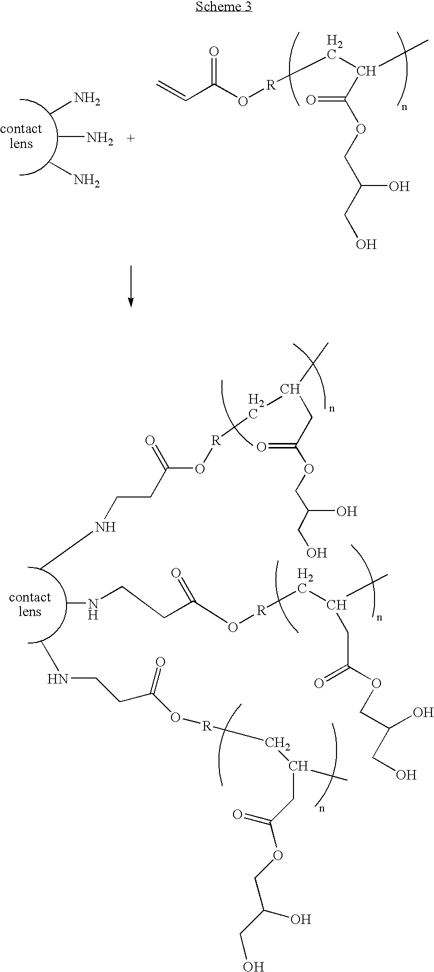

In a further aspect, the medical device comprises a siloxanyl-based polymer. The term “siloxanyl-based” means comprising a silicon-oxygen-silicon bond. Suitable siloxanyl-based polymers are disclosed below.

In still a further aspect, the present invention provides a method of making a medical device that has a hydrophilic and/or lubricious surface. The method comprises: (a) providing the medical device having at least a medical-device surface functional group; (b) providing a macromonomer that comprises at least an ethylenically unsaturated group and a plurality of hydrophilic groups, said at least an ethylenically unsaturated group being capable of reacting with said at least a medical-device surface functional group; and (c) contacting the medical device with the macromonomer at a condition sufficient to produce the medical device having an increased surface hydrophilicity, or lubricity, or both. In one embodiment, said at least an ethylenically unsaturated group is a terminal group. In another embodiment, said at least an ethylenically unsaturated group is a pendant group attached to a unit of the macromonomer.

In one embodiment, the medical-device surface functional groups are parts of a material of the medical device, such as functional groups of a polymeric constituent of the medical device. For example, when the medical device comprises a siloxanyl-based material, such surface functional groups may be provided by ≡SiH or unreacted ≡Si—CH═CH₂ moieties.

In another embodiment, the step of providing the medical device having at least a medical-device surface functional group comprises creating the surface functional group by implantation of moieties that comprise the surface functional group. The implantation is effected at or in the surface of the medical device. In another embodiment, the step of providing the medical device having at least a medical-device surface functional group comprises creating the surface functional group by reacting the material of the surface of the medical device with a suitable reagent to form the surface functional group. In still another embodiment, the suitable reagent is an oxidizing agent. In yet another embodiment, the step of reacting comprises exposing the surface to plasma containing an oxidizing agent, such as an oxygen-containing species, ammonia, amine, or combinations thereof.

In one embodiment of the present invention, the medical-device surface functional groups comprise nitrogen-containing groups.

In another aspect, the medical devices having a coating of the present invention provide higher level of performance quality and/or comfort to the users due to their hydrophilic or lubricious (or both) surfaces. In one embodiment, the medical devices are contact lenses, such as extended-wear contact lenses. Hydrophilic and/or lubricious surfaces of such contact lenses substantially prevent or limit the adsorption of tear lipids and proteins on, and their eventual absorption into, the contact lenses, thus preserving the clarity of the contact lenses, and in turn preserving their performance quality and providing a higher level of comfort to the wearer.

In a further embodiment, the medical device has a polymer coating consisting or consisting essentially of units of N-vinylpyrrolidone.

In one aspect, the surface treatment of the medical device can be carried out, for example, at about room temperature or under autoclave condition. The medical device is immersed in a solution comprising the coating polymer as disclosed above. Alternatively, the medical device is immersed in a solution comprising the coating polymer and a linking compound (or linking polymer). In one aspect, the linking compound has a first linking-compound functional group that is capable of interacting with the medical-device surface functional groups, and a second linking-compound functional group that is capable of interacting with the coating polymer. Thus, in one aspect, the medical device comes into contact with the linking compound and the coating polymer substantially simultaneously. In another aspect, the medical device is immersed in a solution comprising the linking compound. Then, after some elapsed time, the coating polymer is added to the solution in which the medical device is still immersed. In one embodiment of the method of treatment, the solution is aqueous. In another embodiment, the solution comprises a polar organic solvent, such as methanol or ethanol.

In another aspect, the surface of the medical device can be treated with a plasma discharge or corona discharge to increase the population of reactive surface groups. The type of gas introduced into the treatment chamber is selected to provide the desired type of reactive surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate the mercaptan group on the surface. A combination of any of the foregoing gases also can be used in the treatment chamber. Methods and apparatuses for surface treatment by plasma discharge are disclosed in, for example, U.S. Pat. Nos. 6,550,915 and 6,794,456, which are incorporated herein in their entirety by reference. Such a step of treatment with a discharge can be carried out before the treated device is contacted with a medium containing the coating polymer.

Medical devices comprising a wide variety of polymeric materials, including hydrogel and non-hydrogel materials, can be made to have hydrophilic surfaces via a method of the present invention. In general, non-hydrogel materials are hydrophobic polymeric materials that do not contain water in their equilibrium state. Typical non-hydrogel materials comprise silicone acrylics, such as those formed from bulky silicone monomer (e.g., tris(trimethylsiloxy)silylpropyl methacrylate, commonly known as “TRIS” monomer), methacrylate end-capped poly(dimethylsiloxane) prepolymer, or silicones (or polysiloxanes) having fluoroalkyl side groups. On the other hand, hydrogel materials comprise hydrated, cross-linked polymeric systems containing water in an equilibrium state. Hydrogel materials contain about 5 weight percent water or more (up to, for example, about 80 weight percent). Non-limiting examples of materials suitable for the manufacture of medical devices, such as contact lenses, are herein disclosed.

Hydrogel materials for medical devices, such as contact lenses, can comprise a hydrophilic monomer, such as, HEMA, methacrylic acid (“MAA”), acrylic acid (“M”), methacrylamide, acrylamide, N,N′-dimethylmethacrylamide, or N,N′-dimethylacrylamide; copolymers thereof; hydrophilic prepolymers, such as poly(alkylene oxide) having varying chain length, functionalized with polymerizable groups; and/or silicone hydrogels comprising siloxane-containing monomeric units and at least one of the aforementioned hydrophilic monomers and/or prepolymers. Hydrogel materials also can comprise a cyclic lactam, such as N-vinyl-2-pyrrolidone (“NVP”), or derivatives thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

Silicone hydrogels generally have water content greater than about 5 weight percent and more commonly between about 10 to about 80 weight percent. Such materials are usually prepared by polymerizing a mixture containing at least one siloxane-containing monomer and at least one hydrophilic monomer. Typically, either the siloxane-containing monomer or the hydrophilic monomer functions as a crosslinking agent (a crosslinking agent or crosslinker being defined as a monomer having multiple polymerizable functionalities) or a separate crosslinker may be employed. Applicable siloxane-containing monomeric units for use in the formation of silicone hydrogels are known in the art and numerous examples are provided, for example, in U.S. Pat. Nos. 4,136,250; 4,153,641; 4,740,533; 5,034,461; 5,070,215; 5,260,000; 5,310,779; and 5,358,995.

Non-limiting examples of applicable siloxane-containing monomeric units include bulky polysiloxanylalkyl (meth)acrylic monomers. The term “(meth)acrylic” means methacrylic or acrylic, depending on whether the term “meth” is present or absent. An example of bulky polysiloxanylalkyl (meth)acrylic monomers are represented by the following Formula IV:

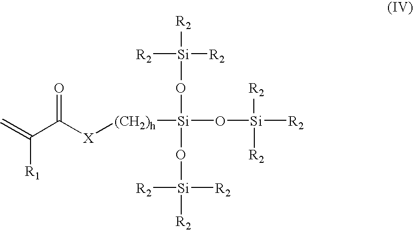

wherein X denotes —O— or —NR—; each R₁ independently denotes hydrogen or methyl; each R₂ independently denotes a lower alkyl radical, phenyl radical or a group represented by

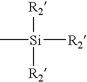

wherein each R′₂ independently denotes a lower alkyl, fluoroalkyl, or phenyl radical; and h is 1 to 10. The term “lower alkyl” means an alkyl radical having 1, 2, 3, 4, 5, 6, 7, 8, 9, or 10 carbon atoms, such as methyl, ethyl, propyl, butyl, isobutyl, pentyl, isopentyl, or hexyl radical.

A suitable bulky monomer is methacryloxypropyltris(trimethyl-siloxy)silane or tris(trimethylsiloxy)silylpropyl methacrylate (“TRIS”).

Another class of representative silicon-containing monomers includes silicone-containing vinyl carbonate or vinyl carbamate monomers such as: 1,3-bis{4-vinyloxycarbonyloxy)but-1-yl}tetramethyldisiloxane; 3-(trimethylsilyl)propyl vinyl carbonate; 3-(vinyloxycarbonylthio)propyl-{tris(trimethylsiloxy)silane}; 3-{tris(trimethylsiloxy)silyl}propyl vinyl carbamate; 3-{tris(trimethylsiloxy)silyl}propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

Another class of representative silicon-containing monomers includes silicone-containing vinyl carbonate or vinyl carbamate monomers such as: 1,3-bis{4-vinyloxycarbonyloxy)but-1-yl}tetramethyl-disiloxane; 3-(trimethylsilyl)propyl vinyl carbonate; 3-(vinyloxycarbonylthio)propyl-{tris(trimethylsiloxy)silane}; 3-{tris(tri-methylsiloxy)silyl}propyl vinyl carbamate; 3-{tris(trimethylsiloxy)silyl}propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula V:

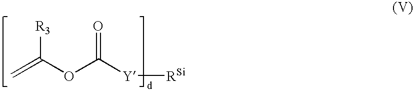

wherein:

Y′ denotes —O—, —S— or —NH—;

R^{Si} denotes a silicon-containing organic radical;

R₃ denotes hydrogen or methyl; and

d is 1, 2, 3 or 4.

Suitable silicon-containing organic radicals R^{Si} include the following:

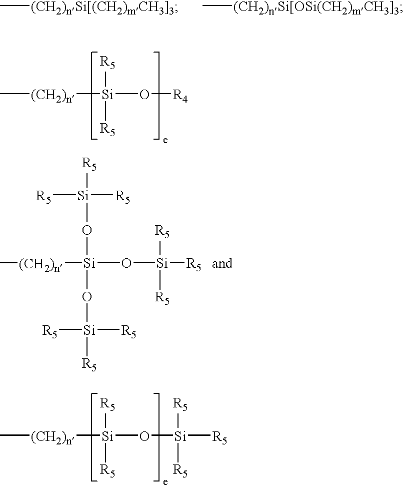

wherein

R₄ denotes

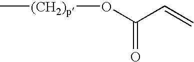

wherein p′ is from 1 to and including 6;

R₅ denotes an alkyl radical or a fluoroalkyl radical having from 1 to and including 6 carbon atoms;

e is 1 to 200; n′ is 1, 2, 3 or 4; and m′ is 0, 1, 2, 3, 4 or 5.

An example of a particular species within Formula II is represented by Formula VI.

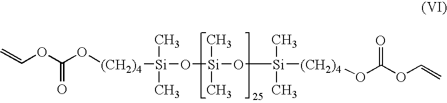

Another class of silicon-containing monomer includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae VII and VIII:

E(*D*A*D*G)ₐ*D*A*D*E′ (VII)

or

E(*D*G*D*A)ₐ*D*G*D*E′ (VII),

wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureylene linkage;

a is at least 1;

A denotes a divalent polymeric radical of Formula IX:

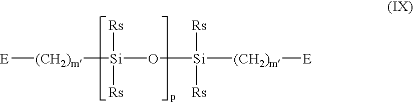

wherein:
each Rₛ independently denotes an alkyl or fluoro-substituted alkyl group
having 1 to 10 carbon atoms which may contain ether linkages between carbon atoms;
m′ is at least 1; and
p is a number which provides a moiety weight of 400 to 10,000;
each of E and E′ independently denotes a polymerizable unsaturated organic radical represented by Formula X:

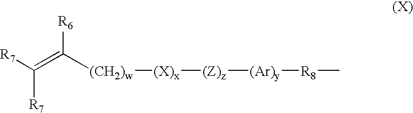

wherein:

R₆ is hydrogen or methyl;

R₇ is hydrogen, an alkyl radical having from 1 to and including 6 carbon atoms, or a —CO—Y—R₉ radical wherein Y is —O—, —S— or —NH—;

R₈ is a divalent alkylene radical having from 1 to and including 10 carbon atoms;

R₉ is a alkyl radical having from 1 to and including 12 carbon atoms;

X denotes —CO— or —OCO—;

Z denotes —O— or —NH—;

Ar denotes a substituted or unsubstituted aromatic radical having from 6 to and including 30 carbon atoms;

w is from 0 to and including 6; x is 0 or 1; y is 0 or 1; and z is 0 or 1.

A more specific example of a silicone-containing urethane monomer is represented by Formula XI:

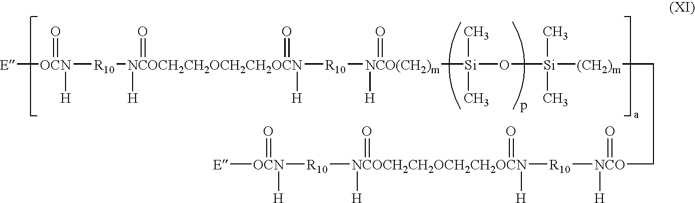

wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

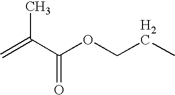

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more poly(siloxanylalkyl(meth)acrylic) monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

In particular regard to contact lenses, the fluorination of certain monomers used in the formation of silicone hydrogels has been indicated to reduce the accumulation of deposits on contact lenses made therefrom, as described in U.S. Pat. Nos. 4,954,587, 5,079,319 and 5,010,141. Moreover, the use of silicone-containing monomers having certain fluorinated side groups (e.g., —(CF₂)—H) have been found to improve compatibility between the hydrophilic and silicone-containing monomeric units, as described in U.S. Pat. Nos. 5,387,662 and 5,321,108.

In another aspect, a polymeric material used to make a polymeric article (such as a medical device) can comprise an additional monomer selected from the group consisting of hydrophilic monomers and hydrophobic monomers.

Hydrophilic monomers can be nonionic monomers, such as 2-hydroxyethyl methacrylate (“HEMA”), 2-hydroxyethyl acrylate (“HEA”), 2-(2-ethoxyethoxy)ethyl(meth)acrylate, glyceryl(meth)acrylate, poly(ethylene glycol (meth)acrylate), tetrahydrofurfuryl(meth)acrylate, (meth)acrylamide, N,N′-dimethylmethacrylamide, N,N′-dimethylacrylamide (“DMA”), N-vinyl-2-pyrrolidone (or other N-vinyl lactams), N-vinyl acetamide, and combinations thereof. Other hydrophilic monomers can have more than one polymerizable group, such as tetraethylene glycol(meth)acrylate, triethylene glycol(meth)acrylate, tripropylene glycol (meth)acrylate, ethoxylated bisphenol-A(meth)acrylate, pentaerythritol (meth)acrylate, pentaerythritol(meth)acrylate, ditrimethylolpropane (meth)acrylate, ethoxylated trimethylolpropane(meth)acrylate, dipentaerythritol (meth)acrylate, alkoxylated glyceryl (meth)acrylate. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process. Non-limiting examples of such substituted anionic hydrophilic monomers include trimethylsilyl esters of (meth)acrylic acid, which are hydrolyzed to regenerate an anionic carboxyl group. The hydrophilic monomer also can be a cationic monomer selected from the group consisting of 3-methacrylamidopropyl-N,N,N-trimethyammonium salts, 2-methacryloyloxyethyl-N,N,N-trimethylammonium salts, and amine-containing monomers, such as 3-methacrylamidopropyl-N,N-dimethyl amine. Other suitable hydrophilic monomers will be apparent to one skilled in the art.

Non-limiting examples of hydrophobic monomers are C₁-C₂₀ alkyl and C₃-C₂₀ cycloalkyl (meth)acrylates, substituted and unsubstituted aryl(meth)acrylates (wherein the aryl group comprises 6 to 36 carbon atoms), (meth)acrylonitrile, styrene, lower alkyl styrene, lower alkyl vinyl ethers, and C₂-C₁₀ perfluoroalkyl(meth)acrylates and correspondingly partially fluorinated (meth)acrylates.

Solvents useful in the surface treatment of the medical device, such as a contact lens, include solvents that readily solubilize the polymers such as water, alcohols, lactams, amides, cyclic ethers, linear ethers, carboxylic acids, and combinations thereof. Preferred solvents include tetrahydrofuran (“THF”), acetonitrile, N,N-dimethyl formamide (“DMF”), and water. The most preferred solvent is water.

### EXAMPLE 1

### Synthesis of Hydroxyl-Containing Poly(vinylpyrrolidone)

To a 1000-ml three-neck flask equipped with a reflux condenser and nitrogen purge inlet tube were added 900 ml of 2-isopropoxyethanol (about 813.6 g, 7.812 mol), 30 ml of distilled NVP (about 31.3 g, 0.282 mol), 0.32 g (1.93 mmol) of AlBN. The contents were bubbled vigorously with nitrogen for 1 hour. While under moderate nitrogen bubbling and stirring, the contents were heated up to 80° C. for two days. The contents were then heated under vacuum to remove the 2-isopropoxyethanol solvent. Hydroxyl-functionalized poly(vinylpyrrolidone) (“PVP”) was obtained, having a number-average molecular weight of greater than about 1300, as determined by titration.

### EXAMPLE 2

### Preparation of Acrylate-Terminated PVP Polymer

To a thoroughly dried 250-ml round-bottom flask equipped with nitrogen purge inlet tube were added, under a flow of dry nitrogen, 16.95 g (12.49 mmol) of hydroxyl-functionalized PVP produced in Example 2 and 200 ml of anhydrous THF in succession. The flask was cooled with an ice bath. Under stirring, 2.72 g (26.88 mmol) of triethylamine was added to the mixture. Acryloyl chloride (2.33g, 25.78 mmol) was added dropwise into the mixture. The reaction mixture was warmed up to room temperature and stirred under dry nitrogen for one day. Deionized water (25 ml) was added to the reaction mixture to give a clear solution. Acrylate-terminated PVP as 16.8% (by weight) solution in methanol was recovered using ultrafiltration to remove low molecular weight species. NMR analysis showed broad acrylate function presence in the polymer.

¹H-NMR: 1H: 6.39 ppm, broad doublet; 6.18 ppm, broad multiple; 5.90 ppm, broad doublet; 4.83 ppm, single; 4.20 ppm, broad multiple; 3.88 ppm, broad single; 3.74 ppm, broad single; 3.34 ppm, single; 3.30 ppm, multiple; from 2.41 ppm˜1.14 ppm, broad multiple; 0.93 ppm, multiple.

### EXAMPLE 3

### Surface Treatment of Silicone-Hydrogel Contact Lenses

PureVision® contact lenses (comprising silicone hydrogel, Bausch & Lomb Incorporated, Rochester, N.Y.) were dried and then plasma treated sequentially with ammonia, butadiene, and ammonia to generate amine-containing groups on the surfaces of the lenses. The lenses were placed in glass vials. Freshly prepared methanol solution containing 16.8% (by weight) of acrylate PVP of Example 2 was then added to the glass vials, which were then set on a rotary machine for three days at room temperature. The treated lenses were rinsed with DI water and stored in borate buffer saline (“BBS”). Control lenses (only plasma treated) were extracted with isopropanol, rinsed with Dl water, and placed in BBS. After being desalinated, both control lenses and coated lenses were subjected to standard surface analysis by XPS, and water contact angles were measured on the lenses. The results are shown in Table 1.

TABLE 1
Comparison Between Treated and Control Lenses

| | Contact | | | | |
|---|---|---|---|---|---|
| | Angle | XPS Analysis (atomic percent) | | | |
| Lenses | (degrees) | C₁ₛ | N₁ₛ | O₁ₛ | Si₂ₚ |
| Control | 79.5 (6.3) | 67.7 (2.3) | 5.8 (0.8) | 18.2 (1.2) | 8.4 (1.4) |
| Coated | 49.1 (4.3) | 76.8 (2.2) | 4.5 (0.4) | 16.1 (0.9) | 2.1 (1.3) |

Note:
The numbers in parentheses are standard deviations of three lenses.

The results show an increase in surface carbon and large decreases in surface nitrogen, oxygen, and silicon, indicating that the lens surfaces are covered with the coating polymer. The water contact angle of coated lenses is smaller than that of control lenses, indicating that the coated lenses are more wettable and, thus, should be more lubricious.

The present invention also provides a method for producing a medical device having improved hydrophilic or lubricious (or both) surfaces. In one aspect, the method comprises: (a) providing the medical device having at least a medical-device surface functional group; (b) providing a coating polymer comprising units of a macromonomer that comprises at least an ethylenically unsaturated group and a plurality of hydrophilic groups, said at least an ethylenically unsaturated group being capable of reacting with said at least a medical-device surface functional group; and (c) contacting the medical device with the polymer at a condition sufficient to produce the medical device having an increased surface hydrophilicity or lubricity or both.

In one aspect, the coating polymer is retained on the surface of the medical device through an interaction of the coating polymer and the medical-device surface functional groups. In another aspect, such an interaction involves complexation or reaction between the coating-polymer functional groups and the medical-device surface functional groups.

In still another aspect, a linking compound (or linking polymer) is provided that has a first linking-compound functional group and a second linking-compound functional group. The first linking-compound functional group interacts with the medical-device surface functional groups, and the second linking-compound functional group interacts with the coating polymer. In one embodiment, such an interaction can be a complexation. In another embodiment, such an interaction can be a formation of chemical bonds.

In a further embodiment, the medical device is contacted with the linking compound or polymer and the coating polymer substantially simultaneously. In another embodiment, the medical device may be contacted with the linking compound or polymer in a medium. The coating polymer is subsequently added into the medium after an elapsed time to produce the finally treated medical device.

The step of contacting can be effected at ambient condition or under autoclave condition at about 120° C. The temperature for treatment can range from ambient to about 120° C., or from slightly above ambient temperature to about 80° C. The treatment time can range from about 10 seconds to about 5 days, or from about 1 minute to about 3 days, or from about 10 minutes to about 24 hours, or from about 10 minutes to about 4 hours, or from about 10 minutes to about 2 hours.

In another aspect, the method further comprises the step of treating the surface of the medical device to increase a population of the medical-device surface functional groups before the step of contacting the medical device with the coating polymer or with the coating polymer and the linking compound or polymer. In still another aspect, the step of treating the surface of the medical device is carried out in a plasma discharge or corona discharge environment. In yet another aspect, a gas is supplied to the discharge environment to provide the desired surface functional groups.

Medical devices having a hydrophilic or lubricious (or both) coating of the present invention can be used advantageously in many medical procedures. For example, contact lenses having a hydrophilic coating of the present invention and/or produced by a method of the present invention can be advantageously used to correct the vision of the natural eye.

In another aspect, the coating polymer of any one of the methods disclosed herein comprises units selected from the group consisting of polymerizable poly(N-vinylpyrrolidone), polymerizable polyhydric alcohols, polymerizable carboxylic acids, copolymers thereof, combinations thereof, and mixtures thereof.

In a further aspect, the present invention provides a method of making a medical device that has reduced affinity for bacterial attachment. The method comprises: (a) forming the medical device comprising a polymeric material; (b) treating the medical device such that a surface thereof becomes more hydrophilic.

In one embodiment, the method comprises: (a) forming the medical device comprising a polymeric material having at least a medical-device surface functional group; (b) contacting the medical device with a coating polymer that comprises units of a macromonomer that comprises both at least an ethylenically unsaturated group and a plurality of hydrophilic groups, said at least an ethylenically unsaturated group being capable of interacting with said at least a medical-device surface functional group to form a coating thereon.

In another embodiment, the interaction between the coating polymer and the surface of the medical device is direct. The coating polymer also may interact indirectly with the surface of the medical device through another compound, such as a linking compound or polymer that comprises a first linking-compound functional group capable of interacting with the medical-device surface functional group and a second linking-compound functional group capable of interacting with the coating polymer. Such interactions can be effected through complexation or chemical reaction or both.

Non-limiting examples of materials for the medical device, the linking compound or polymer, and the coating polymer are disclosed above.

In one embodiment, the medical device is formed by disposing precursors for the medical-device material in a cavity of a mold, which cavity has the shape of the medical device, and polymerizing the precursors.

In another embodiment, a solid block of a polymeric material is first produced, then the medical device is formed from such a solid block; e.g., by shaping, cutting, lathing, machining, or a combination thereof.

In some embodiments, the medical devices produced in a method of the present invention can be contact lenses, intraocular lenses, corneal inlays, corneal rings, or keratoprotheses.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A macromonomer comprising at least an ethylenically unsaturated group and a plurality of hydrophilic groups.

2. The macromonomer of claim 1, wherein the ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group.

3. The macromonomer of claim 1, wherein said at least an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), or crotonate group.

4. The macromonomer of claim 1, wherein said at least an ethylenically unsaturated group comprises vinyl, allyl, or styryl group.

5. The macromonomer of claim 1, wherein the ethylenically unsaturated group is a terminal group.

6. The macromonomer of claim 1, wherein the ethylenically unsaturated group is a pendant group attached to a unit of the macromonomer.

7. The macromonomer of claim 1, wherein said at least an ethylenically unsaturated group is converted to a different functional group.

8. The macromonomer of claim 1, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

9. The macromonomer of claim 2, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

10. The macromonomer of claim 3, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

11. The macromonomer of claim 4, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

12. The macromonomer of claim 1, wherein the plurality of hydrophilic groups comprise N-vinylpyrrolidone.

13. The macromonomer of claim 2, wherein the plurality of hydrophilic groups comprise N-vinylpyrrolidone.

14. A macromonomer comprising an acrylate- or methacrylate-terminated poly(N-vinylpyrrolidone).

15. The macromonomer of claim 14, further comprising units of another hydrophilic monomer.

16. The macromonomer of claim 15, wherein said another hydrophilic monomer is selected from the group consisting of glyceryl(meth)acrylate, dimethyl(meth)acrylamide, 2-hydroxyethyl(meth)acrylate, erythritol(meth)acrylate, xylitol(meth)acrylate, sorbitol(meth)acrylate, derivatives thereof, combinations thereof, and mixtures thereof.

17. A method for making an ethylenically unsaturated hydrophilic macromonomer, the method comprising reacting an oligomer or polymer having a first functional group with a compound having a second functional group that is capable of reacting with the first functional group at a condition sufficient to produce said ethylenically unsaturated hydrophilic macromonomer, wherein the compound comprises an ethylenically unsaturated group and the oligomer or polymer further comprises a plurality of hydrophilic groups.

18. The method of claim 17, wherein the first functional group is a hydroxyl group, the second function group is selected from the group consisting of acryloyl- and methacryloyl-containing halide, isocyanate, and epoxy.

19. A medical device comprising a surface coating that comprises units of a macromonomer that comprises at least an ethylenically unsaturated group and a plurality of hydrophilic groups.

20. The medical device of claim 19, wherein the ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate; itaconate, vinyl, allyl, or styryl group.

21. The medical device of claim 19, wherein said at least an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), or crotonate group.

22. The medical device of claim 19, wherein said at least an ethylenically unsaturated group comprises vinyl, allyl, or styryl group.

23. The medical device of claim 19, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

24. The medical device of claim 20, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

25. The medical device of claim 20, wherein the plurality of hydrophilic groups comprise N-vinylpyrrolidone.

26. The medical device of claim 19, wherein the medical device comprises polysiloxanes.

27. The medical device of claim 26, wherein the medical device comprises hydrogel polysiloxanes.

28. The medical device of claim 19, wherein the medical device is a contact lens, intraocular lens, corneal inlay, corneal ring, keratoprothesis, wound dressing, catheter, or implant.

29. A method for providing increased comfort to a user of a medical device, the method comprising contacting the medical device with a coating material that comprises units of a macromonomer that comprises at least an ethylenically unsaturated group and a plurality of hydrophilic groups.

30. The method of claim 29, wherein the ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group.

31. The method of claim 29, wherein said at least an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), or crotonate group.

32. The method of claim 29, wherein said at least an ethylenically unsaturated group comprises vinyl, allyl, or styryl group.

33. The method of claim 29, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

34. The method of claim 30, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

35. The method of claim 30, wherein the plurality of hydrophilic groups comprise N-vinylpyrrolidone.

36. A method for making a medical device having an increased surface hydrophilicity, or lubricity, or both, the method comprising:
(a) providing the medical device comprising a polymeric material having at least a surface functional group;
(b) providing a coating material that comprises units of a macromonomer that comprises at least an ethylenically unsaturated group and a plurality of hydrophilic groups; and
(c) contacting the medical device with the coating material at a condition sufficient to produce the medical device having an increased surface hydrophilicity, or lubricity, or both.

37. The method of claim 36, wherein the ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), crotonate, maleate, fumarate, itaconate, vinyl, allyl, or styryl group.

38. The method of claim 36, wherein said at least an ethylenically unsaturated group comprises acrylate, methacrylate, sinapate (or 3,5-dimethoxy-4-hydroxycinnamate), cinnamate (or 3-phenylacrylate), senecioate (or 3,3-dimethylacrylate), or crotonate group.

39. The method of claim 36, wherein said at least an ethylenically unsaturated group comprises vinyl, allyl, or styryl group.

40. The method of claim 36, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

41. The method of claim 37, wherein the plurality of hydrophilic groups are selected from the group consisting of N-vinylpyrrolidone, alkylene oxides, glyceryl methacrylate, glyceryl acrylate, dimethyl methacrylamide, dimethyl acrylamide, 2-hydroxyethyl methacrylate, 2-hydroxyethyl acrylate, erythritol methacrylate, erythritol acrylate, xylitol methacrylate, xylitol acrylate, sorbitol methacrylate, sorbitol acrylate, derivatives thereof, combinations thereof, or mixtures thereof.

42. The method of claim 37, wherein the plurality of hydrophilic groups comprise N-vinylpyrrolidone.

43. The method of claim 36, further comprising contacting the medical device with a linking compound in addition to contacting the medical device with the coating material.

44. The method of claim 36, further comprising increasing a population of said at least a surface functional group before the step of contacting.

45. The method of claim 42, wherein said increasing the population of said at least a surface functional group is effected by a method selected from the group consisting of surface implantation of moieties that comprise said functional groups, surface reaction, and combinations thereof.

46. The method of claim 45, wherein the step of increasing the population of said at least a surface functional group is carried out in a plasma discharge or a corona discharge environment.
